(19) United States
(12) Patent Application Publication
(10) Pub. No.: US 2004/0068155 A1
(43) Pub. Date: Apr. 8, 2004

(54) Obtaining anthracene and carbazole by melt-crystallization

(76) Inventors: Thomas Buttner, Wachtberg-Villip (DE); Ulrich Knips, Kamen (DE); Konrad Stolzenberg, Waltrop (DE); J?ouml;rg Talbiersky, Dorsten (DE); Edgar Fuhrmann, Castrop-Rauxel (DE); Friedhelm Alsmeier, Essen (DE); Wolfgang Bergins, Castrop-Rauxel (DE); Siegfried Giertler, Oer-Erkenschwick (DE); Dietmar Scholl, Muhlheim (DE); Bernd Vierhaus, Schwerte (DE); Klaus Dialer, Winterthur (CH); Rudolf Bischof, Walenstadt (CH); Ali Nikzad, Unterengstringen (CH)

Correspondence Address: Andrew S Marks, 47 Rice Road, Wayland, MA, 01778, US

(21) Appl. No.: 10/466,966

(22) PCT Filed: Jan. 23, 2002
(86) PCT No.: PCT/EP02/00605

(30) Foreign Application Priority Data
Jan. 24, 2001 (DE) 101 03 208.0

(51) Int. Cl. C07C 7/14

(52) U.S. Cl. 585/812

(57) ABSTRACT

In a method for obtaining anthracene and carbazole and their sequential products from the anthracene oil resulting during coal tar distillation or its purification products, anthracene oil or the purification product crude anthracene is converted into a melt without adding solvent, the melt is cooled under the crystallization point of carbazole and anthracene, the crystal product obtained is separated from the liquid phase, and the crystal product is distilled to obtain pure anthracene and pure carbazole.

### BACKGROUND OF THE INVENTION

The invention relates to a method for obtaining carbazole and anthracene by melt crystallization.

Carbazole is an intermediate product for the production of dyes, pigments, pesticides, and polymers. The most significant applications for carbazole are violet 23 and hydron blue R. The pigment violet 23 is characterized by high color strength and light-fastness. It is the basis for the production of printing inks and automobile lacquers and for the pigmenting of plastics such as polyvinyl chloride. Hydron blue R is a significant light-fast dye which is extremely well-suited for dying cotton materials. After oxidation to anthraquinone, anthracene is a starting material for the production of anthraquinone dye, whose colorfast properties are characterized as very good. Since the beginning of dye chemistry, they have formed the most important pigment class besides the azo dyes.

The preparation of anthraquinone and carbazole from the anthracene oil of coal tar by crystallization and distillation in the gas phase is described in DE 196 13 497 C1 and is based on the use of solvents in the range from 120 to 210° C. These methods are very expensive due to the high power consumption for redistillation of the solvent.

The catalytic synthesis of carbazole described in DE 19 633 609 A1 cannot compete economically with the preparation of carbazole from coal tar. The preparation of anthracene and carbazole based on the hydrogenation of crude anthracene from coal tar described in DE 757 530 A1 is not currently competitive for reasons of cost. EP 799 813 A1 describes the benzofuran-free preparation of anthracene or of anthraquinone by rectification.

### SUMMARY OF THE INVENTION

The object of the present invention is, starting from coal tar products, such as crude anthracene and anthracene oil, to provide benzofuran-free pure anthracene and a carbazole having perfect color with elevated production output and improved energy efficiency.

This object is achieved by a method for preparing anthracene, carbazole, and possibly sequential products such as anthraquinone from the anthracene oil resulting during coal tar distillation or from crude anthracene, in that a crude anthracene melt or anthracene oil melt is crystallized, and the crystal product is separated from the liquid phase and distilled to obtain pure anthracene and pure carbazole.

With the method according to the invention, anthracene and carbazole are obtained at high purity without the use of solvents and without the low-boiling fractions typically present in anthracene oil.

### DETAILED DESCRIPTION OF THE PREFERRED EMBODIMENTS

The starting material for the method according to the present invention is crude anthracene or anthracene oil. Crude anthracene is obtained from the anthracene oil resulting during coal tar distillation. Coal tar distillation and the preparation of raw anthracene are described, for example, in Franck/Collin, Steinkohlenteer [Coal Tar], Springer-Verlag 1968. Crude anthracene may be prepared from anthracene oil in a known way by cooling crystallization, for example in a agitating crystallizer, and subsequent centrifuging. In this way, crude anthracene 30 is obtained. According to a preferred embodiment of the present invention, crude anthracene is prepared by static melt crystallization. However, the starting material of the method according to present invention may also be anthracene oil. Such anthracene oil may, for example, be withdrawn from the low-boiling oils during the preceding tar distillation.

The anthracene oil is slowly cooled to a temperature of 100 to 20° C. in a crystallizer, preferably a static crystallizer. At the same time, anthracene, carbazole, and phenanthrene crystallize on the surface of the crystallizer. After cooling of the melt to 60 to 20° C., the non-solidified residual oil is drawn off from the crystallizer. The crystal layer located on the crystallizer surface is subsequently slowly heated. During this heating of the crystal product, the impurities, interstitial liquid, and foreign molecules contained in the crystals are melted and drain off from the rest of the crystal layer. This process, referred to as sweating, is continued until a sufficiently large quantity of the crystal product, for example 1 to 20 weight-percent, has drained off. The quantity of oil sweated off may, for example, be observed through the level in the sump of the crystallizer.

Surprisingly, this purification step succeeds even without the solvents typically added and also without the oils such as fluorene and acenaphtene, which lie at the boiling range of 280 to 300° C., containing dibenzofuran, which interferes above all in the production of anthraquinone from anthracene.

After the sweat oil has been drained off, the residual crystal product is completely melted and collected. The composition of this oil corresponds to typically prepared crude anthracene.

This material may be purified in the way described above in further crystallization steps. Depending on the product quality desired, the previously described melt crystallization with the melting is performed two to four more times. At the same time, phenanthrene is separated from the target products anthracene and carbazole, which form a mixed crystal. Preferably, the crystallization and melt temperatures are increased in the further crystallization steps relative to the temperatures of the preceding steps.

It is advantageous that the method according to the invention may be used with a wide range of anthracene oils or crude anthracene. The fractions used may have concentrations of anthracene in a range from 5 to 40 weight-percent, of carbazole in a range from 3 to 25 weight-percent, and a phenanthrene concentration of up to 35 weight-percent. An anthracene-carbazole mixture with concentrations of all attendant materials of 5 to 1% in total is obtained as a product.

The crystal product obtained is subjected in a known way to a distillation, with pure anthracene and pure carbazole being separated from one another. Such a distillation is described, for example, in DE 196 13 497 C1, proceeding, however, from crude anthracene. The distillation of the mixture of anthracene, carbazole and a small proportion of phenanthrene preferably occurs under vacuum, particularly preferably in a packed column, to avoid coatings and discolorations and to minimize energy consumption. The distillation column has at least approximately 60 theoretical plates. The fractions containing the pure anthracene are removed at an upper lateral outlet of the column, and the fractions containing the pure carbazole are removed at a lower lateral outlet. The anthracene fraction boils at a temperature of 335 to 345° C. (normal pressure), and the carbazole fraction at 345 to 370° C. (normal pressure).

To elevate the purity of the anthracene, the hot-liquid anthracene fraction may be recrystallized in a downstream crystallization. At the same time, the last residues of the co-boiling phenanthrene are separated and the purity of the anthracene is elevated to 96 to 99.5% or more. The fraction containing the phenanthrene from this process may be returned to the crystallization to elevate the yield.

The method according to the present invention also allows the use of an anthracene oil with an initial boiling point of more than 300° C. as a starting material. The residual dibenzofuran still included after the melt crystallization may be separated as an overhead product during the subsequent distillation to separate the anthracene/carbazole mixture.

The oxidation of anthracene to anthraquinone may be performed in a known way, for example, by oxygen and hydrogen peroxide. Further methods such as gas phase oxidation are described in H.-G. Franck, J. W. Stadelhofer, Industrielle Aromatenchemie [Industrial Aromatic Chemistry], Springer-Verlag (1987), pp. 358-360.

The following example will explain the invention in more detail without being limited thereto.

### EXAMPLE

An anthracene oil with a content of 7% anthracene, 25.1% phenanthrene, and 3.3% carbazole is brought to a temperature of approximately 200° C. in a heater. The melt obtained is pumped from the heater onto a plate crystallizer. The plates initially also have a temperature of approximately 200° C. and are then cooled. The melt crystallizes on the plates. The plates are then subsequently slightly heated in such a way that a part of the crystallized melt becomes liquid again and drains off. The drained-off melt makes up approximately 10 weight-percent of the materials crystallized on the plates. The portion sweated off is collected and may be recycled in the crude anthracene preparation.

The material remaining on the plates is heated further, liquefied to a melt, and pumped onto the plates of a second crystallizer for further crystallization. The plates initially have a temperature above the melting temperature of the anthracene-carbazole mixture and are then cooled. The melts crystallize out and a proportion of approximately 10% of the crystallized material is again sweated off. The material sweated off in this second crystallization step may be recycled in the first crystallization step.

The material obtained in the second crystallization step after the sweating is melted again and transferred to a third crystallizer. The material is crystallized out and subjected to renewed sweating. The material sweated off may be recycled in the second crystallization step.

The remaining material contains approximately 63% anthracene, 33% carbazole, and 2% phenanthrene.

In comparison to the solvent crystallizations typically used at a significantly lower temperature level (50-150° C.), approximately equal quantities of energy for the heating and cooling processes are necessary in the high temperature melt crystallization according to the present invention. By dispensing with solvent, the masses to be heated and cooled are reduced in such a way that the higher temperatures are not energetically relevant. In the subsequent distillation to separate anthracene and carbazole, the energy consumption is, however, reduced by dispensing with solvent in such a way that the total energy consumption sinks to under 50% of the energy consumption of the typical suspension crystallization.

Now that the preferred embodiments of the present invention have been shown and described in detail, various modifications and improvements thereon will become readily apparent to those skilled in the art. Accordingly, the spirit and scope of the present invention is to be construed broadly and limited only by the appended claims, and not by the foregoing specification.

1. A method for preparing anthracene and carbazole and their sequential products from the anthracene oil resulting during coal tar distillation, or its purification products, characterized in that anthracene oil or its purification product crude anthracene is converted into a melt without adding solvent, the melt is cooled under the crystallization point of carbazole and anthracene, the crystal product obtained is separated from the liquid phase, and the crystal product is distilled to obtain pure anthracene and pure carbazole.

2. The method according to claim 1, characterized in that the melt crystallization is performed in multiple stages.

3. The method according to claim 2, characterized in that the melt crystallization is performed in two stages or in three stages.

4. The method according to one of the claims 1 to 3, characterized in that the melt crystallization is performed on a plate crystallizer.

5. The method according to one of the claims 1 to 4, characterized in that approximately 55 to 65 weight-percent of the melt is allowed to crystallize out on the plates and the residual melt is separated off.

6. The method according to one of the claims 4 or 5, characterized in that the crystal product is caused to sweat on the plates and approximately 10% of the material previously solidified on the plates is allowed to drain off and the plates are cooled again.

7. The method according to one of the claims 1 to 6, characterized in that an anthracene oil with an initial boiling point at a temperature of more than 300° C. is used as a starting material.

8. The method according to claim 7, characterized in that residual dibenzofuran is separated as an overhead product during the separation of the anthracene-carbazole mixture by distillation.

9. The method according to claim 1 or 8, characterized in that the anthracene fraction obtained by distillation is recrystallized.

10. The method according to one of the claims 1 to 9, characterized in that the pure anthracene obtained is oxidized to anthraquinone.

11. The method according to one of the claims 1 to 10, characterized in that the pure carbazole, pure anthracene, or anthraquinone obtained is granulated, possibly milled again, and packaged.
